(19) United States
(12) Patent Application Publication
(10) Pub. No.: US 2008/0183238 A1
(43) Pub. Date: Jul. 31, 2008

(54) PROCESS FOR ELECTROSTIMULATION TREATMENT OF MORBID OBESITY

(75) Inventor: Jiande Chen, Houston, TX (US)

Correspondence Address: SHUMAKER & SIEFFERT , P.A, 1625 RADIO DRIVE , SUITE 300, WOODBURY, MN, 55125, US

(73) Assignee: Medtronic, Inc., Minneapolis, MN (US)

(21) Appl. No.: 12/023,809

(22) Filed: Jan. 31, 2008

Related U.S. Application Data
(62) Division of application No. 10/627,908, filed on Jul. 25, 2003.
(60) Provisional application No. 60/398,886, filed on Jul. 26, 2002.

(51) Int. Cl. A61N 1/05

(52) U.S. Cl. 607/40

(57) ABSTRACT

An improved process using electrostimulation for treating obesity, especially morbid obesity, is provided. The improved method of this invention provides electrostimulation on or along the small intestines, preferably on or along the duodenum and/or jejunum, which provides improved control of obesity. In one embodiment, the process employs stimulation of the lesser curvature at a rate of about 2 to about 30 pulses/minute with each pulse lasting about 0.1 to about 4 seconds such that there is a pause of about 3 to about 30 seconds between the pulses. More preferably, the pulse rate is about 12 to about 14 pulses/minute with each pulse lasting about 0.1 to about 0.5 seconds with a pause of about 4.5 to about 5 seconds between pulses. Preferably, the pulse amplitude is about 0.5 to about 15 milliamps. More preferable, each pulse consists of a train of micro-bursts with a frequency of about 5 to about 100 Hz.

### RELATED APPLICATIONS

This application is based on, and claims benefit of, U.S. Provisional Application Ser. No. 60/398,886, filed on Jul. 26, 2002, which is hereby incorporated by reference.

### FIELD OF THE INVENTION

The present invention relates to an improved process using electrostimulation for treating obesity, especially morbid obesity, and other syndromes related to motor disorders of the stomach. The improved method of this invention provides electrostimulation on, or adjacent to, the small intestines which provides improved control of obesity and other syndromes related to motor disorders of the stomach. Duodenal electrical stimulation is especially preferred.

### BACKGROUND OF THE INVENTION

The modern surgical orientation with regard to obesity generally entails the reduction of gastric compliance, with the aim of limiting the subject's ability to ingest food, or of reducing the food absorption surface by shortening or bypassing part of the digestive canal; both aims are sought in some surgical procedures. Until recently, surgery was the only therapy that ensures real results in patients who have exceeded obesity values close to or greater than about 40 BMI (ratio of weight in kilograms to the square of the height in meters).

All of the major surgical procedures (e.g., removal or blocking off of a portion of the stomach) currently in use have some immediate and/or delayed risks. Thus, surgery is usually considered as an extreme solution when all less invasive procedures fail. Furthermore, even surgical treatment fails in some cases, thereby requiring the surgeon to restore the original anatomical situation.

More recently, methods have been successfully employed whereby an electrostimulation device is implanted on the stomach wall. For example, U.S. Pat. No. 5,423,872 (Jun. 13, 1995) provided a process for the treatment of obesity and related disorder employing an electrostimulator or pacemaker attached to the antrum or greater curvature of the stomach. U.S. Pat. No. 5,690,691 (Nov. 25, 1997) provided a portable or implantable gastric pacemaker including multiple electrodes positionable on the inner or outer surface of an organ in the gastrointestinal tract which are individually programmed to deliver a phased electrical stimulation to pace peristaltic movement of material through the gastro-intestinal tract. Although these methods have generally been successful, it is still desirable to provide improved methods for such treatments. The present invention provides such an improved process.

### SUMMARY OF THE INVENTION

The present invention provides a process for treating obesity and/or related motor disorders by providing at least one electrostimulation or pacemaker device attached to, or adjacent to, the small intestines or lower bowel. Duodenal electrical stimulation is especially preferred. The electrostimulation may include relatively long pulses or pulse trains (i.e., microbursts). Preferably, the process of this invention employs stimulation of the duodenum and/or the jejunum. Preferably the individual pulses are at a rate of about 2 to about 30 pulses/minute with each pulse lasting about 0.1 to about 4 seconds such that there is a pause of about 3 to about 30 seconds between the pulses. More preferably, the pulse rate is about 12 to about 14 pulses/minute with each pulse lasting about 0.1 to about 0.5 seconds with a pause of about 4.5 to about 5 seconds between pulses. Preferably, the pulse amplitude is about 0.5 to about 15 milliamps. More preferable, electrostimulation in the form of a train of micro-bursts (see FIG. 2) with a frequency of about 10 to about 100 Hz, and more preferably of about 40 Hz.

The process of the present invention involves treatment of obesity and other syndromes related to motor disorders of the stomach of a patient. The process comprises artificially altering, using sequential electrical pulses for preset periods of time, the natural gastric motility of the patient to prevent or slow down stomach emptying, thereby slowing food transit through the digestive system. Although not wishing to be limited by theory, stimulation of the lower intestines appears to result in an expansion of the stomach and, due to a feeling of satiation, reduced intake of food. Again not wishing to be limited by theory, intestinal stimulation appears to lead to secretion (and/or increased secretion) of gastrointestinal peptides which may inhibit gastrointestinal motility and induce satiety. Again not wishing to be limited by theory, intestinal stimulation also appears to accelerate intestinal transit and thus reduce absorption time within the intestinal tract.

The present invention provides a method for treatment of a motor disorder of a patient's stomach, said method comprising implanting at least one electrostimulation device comprising one or more electrostimulation leads and an electrical connector for attachment to a pulse generator such that the one or more electrostimulation leads are attached to, or adjacent to, small intestines, whereby electrical stimulation can be provided to the small intestines through the one or more electrostimulation leads; and supplying electrical stimulation to the small intestines through the one or more electrostimulation leads.

### BRIEF DESCRIPTION OF THE DRAWING

FIG. 1A is a sectional view of the stomach. FIG. 1B is a sectional view of a gastrointestinal tract showing the device of the invention in place along the small intestines.

FIG. 2 is a schematic representation (not to scale) of a preferred microburst pulse train provided to the small intestines.

### DETAILED DESCRIPTION OF THE PREFERRED EMBODIMENTS

The present invention provides a process for treating obesity and/or related motor disorders by providing an electrostimulation or pacemaker device attached to, or adjacent to, the small intestines such that the small intestines may be electrostimulated. Generally, electrostimulation of the duodenum and/or the jejunum is generally preferred with electrostimulation of the duodenum being especially preferred. In an especially preferred embodiment, electrostimulation of both the duodenum and/or the jejunum is preferred.

Preferably, the process of this invention employs stimulation of the lower intestines at a rate of about 2 to about 30 pulses/minute with each pulse lasting about 0.1 to about 4 seconds such that there is a pause of about 3 to about 30 seconds between the pulses. More preferably, the pulse rate is about 12 to about 14 pulses/minute with each pulse lasting about 0.1 to about 0.5 seconds with a pause of about 4.5 to about 5 seconds between pulses. Preferably, the pulse amplitude is about 0.5 to about 15 milliamps. More preferable, each pulse consists of a train of micro-bursts with a frequency of about 5 to about 100 Hz.

The process of the present invention involves treatment of obesity and other syndromes related to motor disorders of the stomach of a patient. The process comprises artificially altering, using sequential electrical pulses for preset periods of time directed to the small intestines, thereby decreasing food intake. Electrostimulation of the small intestines may also prevent or slow down stomach emptying, thereby slowing food transit through the digestive system, and contributing to the feeling of satiety in the patient. Although not wishing to be limited by theory, it is thought that this improvement is at least in part due to inhibitory biofeedback mechanisms between the small intestines and the stomach.

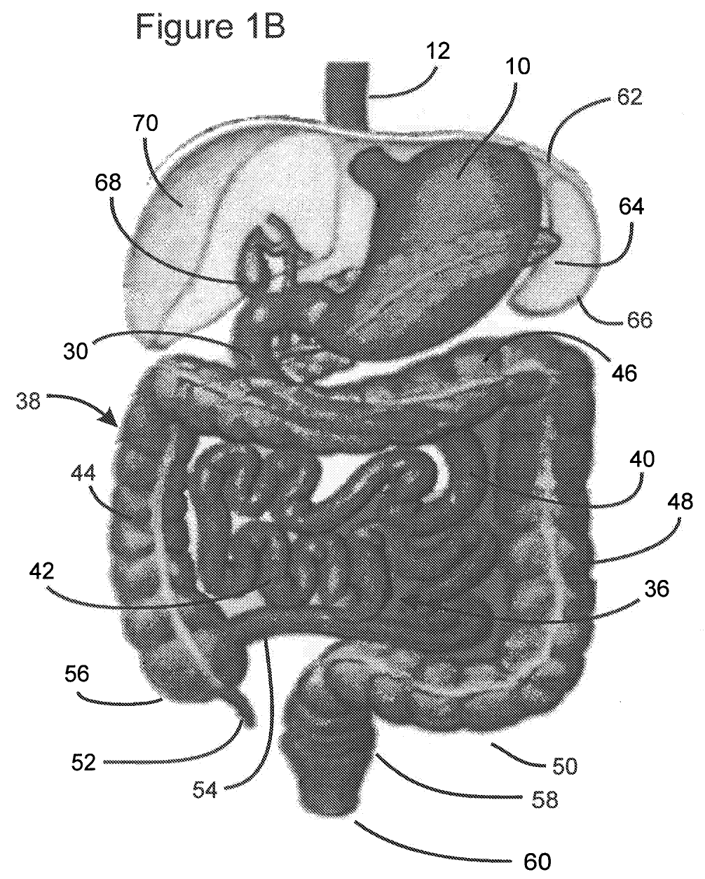

The method of this invention provides electrostimulation to the small intestines; preferably electrostimulation is applied to at least two locations on the small intestines. Electrical stimulus may consist of single pulses or pulse trains. Generally, single pluses have relatively long durations (i.e., about 10 ms to about 600 ms) are preferred. Preferably, the frequency of the stimulation preferably will be similar to the frequency of intestinal slow waves (about 12 cycles/min (cpm) in human duodenal and about 8 to about 9 cpm in the ileum). Thus, the frequency is preferably in a range of about 8 to about 30 cpm. The stimulus may also be in a form of pulse trains or microbursts with an internal frequency of about 10 to 100 Hz (see FIG. 2).

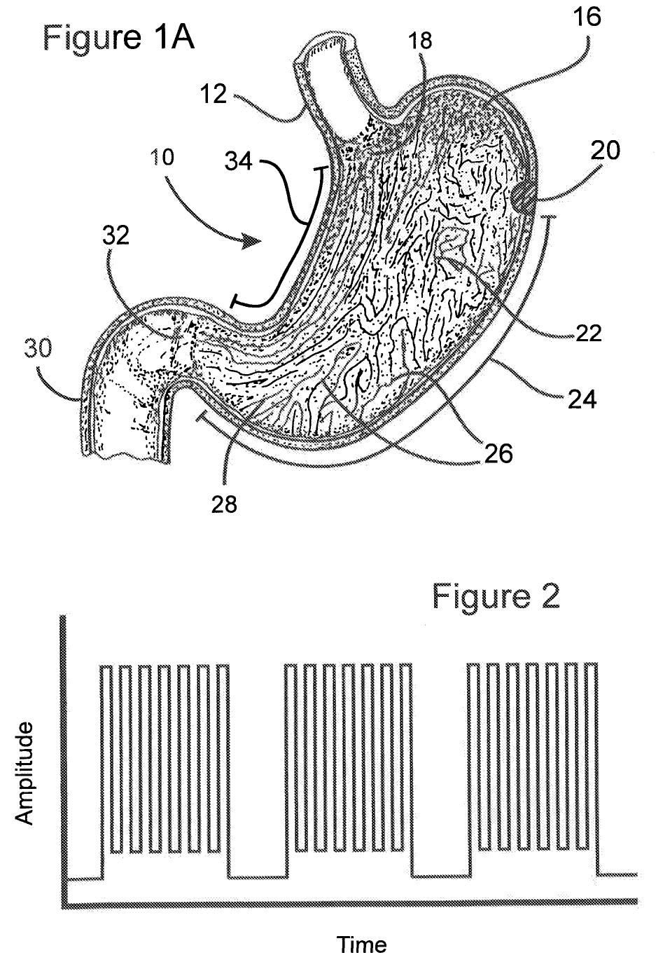

In order to further clarify the process and device for treating obesity and syndromes related to motor disorders of the stomach of a patient, according to the invention, the motor physiology of the gastric viscus is briefly described. FIGS. 1A and 1B, respectively, illustrate the stomach and the general gastrointestinal tract. As shown in FIG. 1A, the stomach 10 is supplied by the esophagus 12, and has the fundus ventriculi 16, the cardia 18, the body or corpus ventriculi 22, the antrum 28, the pylorus 32, the duodenum 30 (i.e., the initial portion of the small intestines), and mucous folds or rugae 26. The lesser curve 34 and greater curve 24 are also shown. The stomach 10 is generally divided into two parts as regards its motility: the fundus ventriculi 16, which has tonic wall movements, and the central part or corpus 22, which is characterized by phasic activity. Propulsive gastric movements begin at a point proximate to the greater curvature 24 which is not clearly identified anatomically and is termed “gastric pacemaker” 20. The gastric pacemaker 20 sends electrical pulses (depolarization potential) at a rate of approximately three times per minute which spread in an anterograde direction along the entire stomach in the form of waves.

The antrum 28 of the stomach has a continuous phasic activity which has the purpose of mixing the food which is present in the stomach. The passage of food into the duodenum 30 is the result of a motility coordinated among the antrum 28, pylorus 32, and duodenum 30. The gastric pacemaker 20 spontaneously and naturally generates sinusoidal waves along the entire stomach; these waves allow the antrum 28, in coordination with the pylorus 32 and duodenum 30, to allow food to pass into the subsequent portions of the alimentary canal (i.e., small intestines 40 and later large intestines 38 in FIG. 1B).

As shown in FIG. 1B, the small intestines 40 generally consist of duodenum 30, jejunum 40, and ileum 42; the enzymatic digestion and essentially all absorption occurs in the small intestines. The ileum 42 empties into the large intestines 38 via the ileocecal valve 54. The major features of the large intestines 38 include the cecum 56, appendix, 52, ascending colon 44, transverse colon 46, descending colon 48, sigmoid colon 50, anal canal 58, and finally the anus 60. Also shown for completeness is the diaphragm 62, spleen 66, pancreas 64, gallbaldder 68, and liver 70.

Now that the known physiology of the gastric motility of a mammal, such as a human being, has been established, the process according to the invention consists in artificially altering, by means of sequential electrical pulses and for preset periods of time, the natural gastric motility of a patient by electrostimulation of the small intestines or lower bowel. More particularly, the sequential electrical pulses are generated by an electrical stimulator which is applied by laparoscopic means to a portion of, or adjacent to, the small intestines. Preferred locations for electrostimulation include along the duodenum 30 and the jejunum 40. Of course, other portions of the small intestines 36 can be electrostimulated using the method of this invention.

The stimulator can be programmed both for continuous stimulation and for “on demand” stimulation (i.e., at the onset of a particular electrical activity which can be detected by the stimulator itself through the electrocatheter (if modified to monitor electrical activity) or under the control of the patient or medical personnel).

The electrical stimulator preferably has a preset operating frequency and period which may obviously vary according to the alteration of stomach motility to be obtained and/or to the pathological condition of the patient. Generally, the electrical stimulator has an operating frequency of about 2 to about 30 pulses per minute. Preferably, the process of this invention employs stimulation of the small intestines at a rate of about 2 to about 30 pulses/minute with each pulse lasting about 0.1 to about 4 seconds such that there is a pause of about 3 to about 30 seconds between the pulses. The electrical discharge of each pulse can vary from approximately 1 to 15 volts for voltage-controlled stimulation and from 2 to 15 milliamperes for constant current stimulation. More preferably, the pulse rate is about 12 to about 14 pulses/minute with each pulse lasting about 0.1 to about 0.5 seconds with a pause of about 4.5 to about 5 seconds between pulses. Preferably, the pulse amplitude is about 0.5 to about 15 milliamps. More preferable, each pulse consists of a train of micro-bursts with a frequency of about 5 to about 100 Hz. FIG. 2 generally illustrates a preferred microburst pulse train provided to the lower intestines.

The present invention generally uses conventional laparoscopic or minimally invasive surgical techniques to place the desired electrostimulation device or devices on, or adjacent to, the small intestines 36, whereby electrostimulation of the small intestines 36 can be effected. Conventional electrostimulation devices may be used in the practice of this invention. Such devices include, for example, those described in U.S. Pat. No. 5,423,872 (Jun. 3, 1995) (an implantable gastric electrical stimulator at the antrum area of the stomach which generates sequential electrical pulses to stimulate the entire stomach, thereby artificially altering the natural gastric motility to prevent emptying or to slow down food transit through the stomach); U.S. Pat. No. 5,690,691 (Nov. 25, 1997) (a portable or implantable gastric pacemaker employing a number of electrodes along the greater curvature of the stomach for delivering phased electrical stimulation at different locations to accelerate or attenuate peristaltic movement in the GI tract); U.S. Pat. No. 5,836,994 (Nov. 17, 1998) (an implantable gastric stimulator which incorporates direct sensing of the intrinsic gastric electrical activity by one or more sensors of predetermined frequency bandwidth for application or cessation of stimulation based on the amount of sensed activity); U.S. Pat. No. 5,861,014 (Jan. 19, 1999) (an implantable gastric stimulator for sensing abnormal electrical activity of the gastrointestinal tract so as to provide electrical stimulation for a preset time period or for the duration of the abnormal electrical activity to treat gastric rhythm abnormalities); PCT Application Serial Number PCT/US98/10402 (filed May 21, 1998) and U.S. patent application Ser. No. 09/424,324 (filed Jan. 26, 2000) (implant device equipped with tines to help secure it in the appropriate location); U.S. Pat. No. 6,041,258 (Mar. 21, 2000) (electrostimulation device with improved handle for laparoscopic surgery); U.S. patent application Ser. No. 09/640,201 (filed Aug. 16, 2000) (electrostimulation device attachable to enteric or endo-abdominal tissue or viscera which is resistance to detachment); PCT Application Serial Number PCT/US00/09910 (filed Apr. 14, 2000; Attorney Docket Number 3581/006 PCT) entitled “Gastric Stimulator Apparatus and Method for Installing” based on U.S. Provisional Application Ser. Nos. 60/129,198 and 60/129,199 (both filed Apr. 14, 1999); PCT Application Serial Number PCT/US00/10154 (filed Apr. 14, 2000; Attorney Docket Number 3581/004 PCT) entitled “Gastric Stimulator Apparatus and Method for Use” based on U.S. Provisional Application Ser. Nos. 60/129,209 (filed Apr. 14, 1999) and 60/466,387 (filed Dec. 17, 1999); and U.S. Provisional Patent Application Ser. No. 60/235,660 (filed Sep. 26, 2000) entitled “Method and Apparatus for Intentional Impairment of Gastric Motility and/or Efficiency by Triggered Electrical Stimulation of the Gastric Tract with Respect to the Intrinsic Gastric Electrical Activity.” All of these patents, patent applications, provisional patent applications, and/or publications are hereby incorporated by reference.

Preferred electrostimulation devices include electrocatheters having an elongated body with a distal end having an electrostimulation lead or leads mounted on, or attached to, the stomach in the region of the lesser curvature and a proximal end for attachment to a pulse generator. The electrostimulation lead or leads are attached to a power source through, or with, the pulse generator. Such preferred electrostimulation devices are described in, for example, PCT Application Serial Number PCT/US98/10402 (filed May 21, 1998), U.S. patent application Ser. No. 09/424,324 (filed Jan. 26, 2000), and U.S. patent application Ser. No. 09/640,201 (filed Aug. 16, 2000). Of course, care should be taken in placement or attachment of the electrostimulation device to avoid physical strangulation of the small intestines.

The present methods can also be used in combination with electrostimulation of other parts of the gastrointestinal tract. For example, electrostimulation could be applied to the small intestines as well as one or more location within the gastrointestinal tract. The sites of electrostimulation could be phased or non-phased in relation to one another.

The following examples are provided to describe the invention and not to limit it.

### EXAMPLE 1

This Example illustrates the duodenal electrical stimulation (DES) on acute food intake. Each of 8 healthy dogs was equipped with a gastric cannula for the measurement of gastric tone and one pair of bipolar electrodes on duodenal serosa. Session without DES (control) and session with DES (inventive method) were carried out. The DES sessions used single pulses repeated at 10 pulses/min; the pulses had a pulse width of 334 ms and pulse amplitude of 6 mA. After a 28 hour fast, the subject dogs were given unlimited access to solid food and water for 1 hour with or without DES. The experiment was repeated in 4 vagotomized (truncal) dogs. In similar studies, the gastric volume at a fixed pressure was measured using a computerized barostat device for 30-min at baseline, 30-min with DES, and 30-min after DES.

DES significantly reduced food intake in both intact dogs (344±38 g in DES-treated subjects as compared to 487±34 g in the controls (p=0.001)) and in vagotomized subjects (137±109 g in DES-treated subjects as compared to 448±72 g in the controls (p=0.02)). Water intake was essentially the same in all subjects.

The gastric volume measurements demonstrated that DES significantly relaxed the stomach. The gastric volume was 321±37 ml at baseline, increased to 439±29 ml during DES (p=0.04), and returned to 358±48 ml after DES.

Thus, DES substantially reduces food intake and, therefore, should be effective in treatment of obesity. Although not wishing to be limited by theory, this inhibitory effect does not appear to be vagally mediated but possibly may be attributed to the induced relaxation of the stomach.

### EXAMPLE 2

This experiment was performed on Sprague-Dawley rats under anesthesia. Four groups of ten rats were subjected to the following experiments: Group 1: control group—no electrical stimulation; Group 2: intestinal electrical stimulation with long pulses (28 pulses/min at 200 ms and 4 mA); Group 3: intestinal electrical stimulation with pulse train (2 seconds on, 3 seconds off; 40 Hz at 2 ms pulse width and 4 mA pulse amplitude); and Group 4: intestinal electrical stimulation with pulse train (same pulse/stimulation parameters as Group 3) plus lidocaine (0.5 mg in 10 ml saline dropped onto intestinal serosal during electrical stimulation).

For each group, a fat solution (triglyceride) was perfused via a catheter inserted into the proximal jejunum and then collected from another catheter inserted into the distal jejunum during a 45 minute test duration. Fat absorption was estimated by the difference between the total perfused fat and the total collected fat at the distal jejunum. For electrical stimulation (Groups 24), a pair of serosal electrodes were implanted on the proximal jejunum and activated during perfusion with the fat solution. The average total fat absorbed for the four groups over the 45 minute test period was as follows: Group 1—about 37 percent; Group 2—about 21 percent (p<0.05 as compared to control Group 1); Group 3—about 6 percent (p<0.001 as compared to either control Group 1 or long pulse Group 2); and Group 4—about 24 percent (p<0.05 as compared to control Group 1; p<0.01 as compared to pulse train Group 3). Thus, a substantial and significant decrease in fat absorption due to electrical stimulation was observed. The partial blockage of the effect of pulse train stimulation by lidocaine (Group 4 as compared to Group 3) suggests the involvement of enteric nerves in the pulse train electrical stimulation.

The methods and electrostimulators used in the present invention are susceptible to numerous modifications and variations, all of which are within the scope of the present inventive concept. Furthermore, all the details may be replaced with technically equivalent elements. The materials employed, the shapes, and the dimensions of the specific electrostimulators may be varied according to the requirements.

1-19. (canceled)

20. A method for treatment of obesity in a patient, the method comprising:
delivering electrical stimulation from an electrostimulation device to a small intestine of a patient via one or more implanted electrostimulation leads,
wherein the electrical stimulation of the small intestine is configured such that the electrical stimulation of the small intestine causes an increase in volume of a stomach of the patient to substantially reduce food intake by the patient.

21. The method of claim 20, further comprising delivering the electrical stimulation to the duodenum of the patient.

22. The method of claim 20, further comprising delivering the electrical stimulation to both the duodenum and a jejunum of the patient.

23. The method of claim 20, further comprising delivering the electrical stimulation to at least two locations of a small intestine of the patient.

24. The method of claim 20, further comprising delivering the electrical stimulation in response to an onset of electrical activity detected by the stimulator.

25. The method of claim 20, further comprising delivering the electrical stimulation in response to user input.

26. The method of claim 20, further comprising delivering the electrical stimulation supplied to the small intestines at a rate of about 2 to about 30 pulses per minute.

27. An electrostimulation device for treatment of obesity in a patient the device comprising:
an electrical stimulation generator that generates electrical stimulation; and
at least one implantable electrical stimulation lead coupled to the stimulation generator to allow delivery of the electrical stimulation to a small intestine of the patient,
wherein the electrical stimulation is configured to stimulate the small intestine such that the stimulation causes an increase in volume of a stomach of the patient to substantially reduce food intake by the patient.

28. The device of claim 27, wherein the electrical stimulation is configured to stimulate the duodenum of the patient such that the stimulation causes the increase in volume of the stomach of the patient to substantially reduce food intake by the patient.

29. The device of claim 27, wherein the electrical stimulation is configured to stimulate both the duodenum and a jejunum of the patient such that the stimulation causes the increase in volume of the stomach of the patient to substantially reduce food intake by the patient.

30. The device of claim 27, wherein the device is configured to deliver the electrical stimulation to at least two locations of a small intestine of the patient.

31. The device of claim 27, wherein the device is configured to deliver the electrical stimulation in response to an onset of electrical activity detected by the stimulator.

32. The device of claim 27, wherein the device is configured to deliver the electrical stimulation in response to user input.

33. The device of claim 27, wherein the device is configured to deliver the electrical stimulation to the small intestines at a rate of about 2 to about 30 pulses per minute.
